(19) United States
(12) Patent Application Publication
(10) Pub. No.: US 2023/0341518 A1
(43) Pub. Date: Oct. 26, 2023

(54) MILLIMETERWAVE RADAR SYSTEM FOR DETERMINING AN ACTIVITY RECORD

(71) Applicant: NODENS MEDICAL LTD., London (GB)

(72) Inventors: Khalid RAJAB, London (GB); Rishi DAS-GUPTA, London (GB); Peter ALIZADEH, London (GB)

(21) Appl. No.: 18/026,660

(22) PCT Filed: Sep. 16, 2021
(86) PCT No.: PCT/GB2021/052409

§ 371 Date: Mar. 16, 2023

(30) Foreign Application Priority Data
Sep. 16, 2020 (GB) 2014577.7
Jun. 30, 2021 (GB) 2109457.8

(51) Int. Cl. G01S 7/41; G01S 7/00; A61B 5/05; A61B 5/00; A61B 5/11

(52) U.S. Cl. CPC G01S 7/415; G01S 7/417; G01S 7/003; A61B 5/05; A61B 5/7264; A61B 5/7275; A61B 5/1118; A61B 2562/0219; A61B 5/4815; A61B 5/1117

(57) ABSTRACT

There is provided a mm-wave radar system for detecting an activity record from multiple targets. At least one sensor is configured to transmit a mm-wave signal waveform and to receive backscattered signals from multiple targets. One or more processors configured to process the received backscattered signals, determine radar data related to each target, and process the radar data of each target using a machine learning (ML) engine that outputs an activity record related to each target. Each activity record is linked to a specific timestamp.

### BACKGROUND OF THE INVENTION

### 1. Field of the Invention

The field of the invention relates to a millimeter wave radar system for determining an activity record and to related methods and sensor devices.

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### 2. Description of the Prior Art

Radars operating in the radio frequency (RF) and millimeterwave (mm-wave) bands present the ability to track and monitor position and movement in both indoor and outdoor environments. A wide range of applications exists. However, the number of specific use cases that have been implemented is still limited.

Microwave radars have been used previously for detection of vital signs, through analysis of signal amplitude or phase. However they have not been used for long-term monitoring, prediction, and evaluation of physical wellbeing.

Known solutions for subject behavior, such as fall detection or prediction have either used video-based techniques or wearable devices including sensor suites such as gyroscopes or accelerometers. There is a need for a less intrusive solution.

Further, detecting multiple moving targets is a challenge, as dynamic scenes with a lot of motion leads to clutter and noise, which interfere with the responses of targets of interest. Additionally, complex indoor environments can clutter scenes, leading to false detections or missed readings.

The present invention addresses the above vulnerabilities and also other problems not described above.

### SUMMARY OF THE INVENTION

One aspect of the invention relates to a mm-wave radar system for detecting an activity record from multiple targets comprising:
i) at least one sensor configured to transmit a mm-wave signal waveform and to receive backscattered signals from multiple targets; and
ii) one or more processors configured to a) process the received backscattered signals, b) determine radar data related to each target, and c) process the radar data of each target using a machine learning (ML) engine that outputs an activity record related to each target, in which each activity record is linked to a timestamp.

### BRIEF DESCRIPTION OF THE FIGURES

Aspects of the invention will now be described, by way of example(s), with reference to the following Figures, which each show features of a mm-wave radar system that implements the invention:

FIG. 1 shows a block diagram with an example of sensor architecture.

FIG. 2 shows a simplified block diagram with an example of the overall system architecture is shown.

FIG. 3 shows a MIMO processing scheme.

FIG. 4 shows an illustration of a DBSCAN clustering algorithm applied to detect two clusters.

FIG. 5 shows an illustration of the efficacy of the beamforming-based approach in separating responses of two equidistant targets.

FIG. 6 shows the micro-Doppler signatures obtained over a 1.5 s interval, for six different activities.

### DETAILED DESCRIPTION

Millimeterwave (mm-wave) radar coupled with advanced processing techniques, including a spatial filtering-based approach and machine learning, is used to make high-resolution tracking, activity classification, and vital signs detection possible, all at low cost, without the use of wearable devices, and at higher precisions than is possible with most other wireless approaches. This is due in part because of the shorter wavelengths, and larger multi-gigahertz bandwidths available, principally at around 60 GHz, which is unlicensed in many regions across the globe.

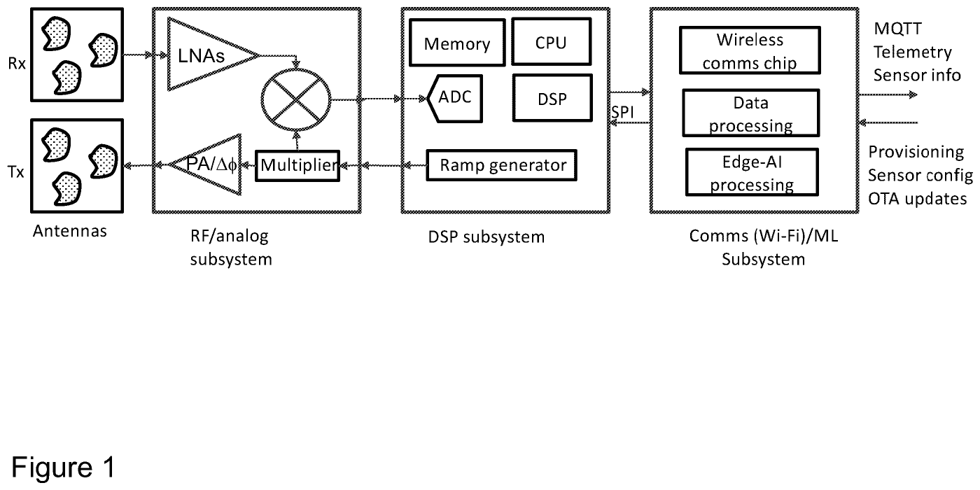

With reference to FIG. 1, a block diagram with an example of sensor architecture is shown, including transmitting (Tx) and receiving (Rx) antennas, an RF/analog subsystem, a digital signal processing (DSP) subsystem, and a communications (Comms)/machine learning subsystem.

The system is composed of one, or a plurality, of mm-wave radars or sensors, each of which contains an on-board microprocessor comprising the necessary analog electronics to generate mm-wave signals (including but not limited to a waveform generator, voltage-controlled oscillator, linear and power amplifiers, multipliers, phase shifters), to transmit and receive the signals (transmit and receive antennas and their respective delay lines), as well as an analog-to-digital/digital-to-analog converter, and the necessary digital electronics to generate the waveforms and process the returned radar signals (including but not limited to microprocessors, memory). A waveform is generated in the digital domain, and then converted to analog, mixed to the mm-wave frequencies, amplified and transmitted. The returned signal is then mixed down to baseband, digitized and analyzed through radar signal processing and other digital processing techniques. The radar is based on a frequency modulated continuous wave (FMCW) architecture, and signal modulation types could include time division multiplexing (TDM) or binary pulse modulation (BPM), depending on noise and processing requirements.

The returned signals, in the form of complex in-phase and quadrature (IQ) components, are further processed using the on-board microprocessor, to obtain a radar data cube. The microprocessor stores the radar data cube (RDC) or a portion of the RDC. The size of the complex-valued RDC is given as,

RDC size=2×data type(bytes)×N_{Rx}×N_{Tx}×N_{chirp}×N_{adc}. (1)

For a 32-bit data type, with N_{Rx}=4 receive antennas, N_{Tx}=3 transmit antennas, N_{chirp}=128 chirps per frame, and N_{adc}=128 ADC samples per chirp, the RDC will have size 1,572,864 bytes. At a common frame rate of 20 fps, which is suitable for capturing human motions and micro-movements, a data transfer rate of 30 MB/s is necessitated. This is excessive, both from the perspective of the data pipeline as it would be challenging for most wireless transmission protocols (Wi-Fi, etc.), and because data storage and transmission off-site (especially on commercial Cloud storage facilities) would be expensive. Therefore, it is preferable to reduce the data size on-chip (i.e., on the edge). Algorithmic radar signal processing approaches exist to estimate many parameters, such as positions and velocities of targets (people and objects), as well as to remove clutter and to track the targets. Machine learning approaches, including but not limited to k-nearest neighbors (KNN), k-means clustering, multi-level perceptron (MLP), and artificial neural networks (ANN) can be used to classify activities, however it is challenging to do this accurately on-chip as these techniques tend to be computationally expensive.

There are various approaches to accurately classifying activities in sensor and Internet-of-Things (IoT) systems, ranging from embedding higher-power processors on-chip (multi-core CPU, GPUs, TPU, etc.), to transmitting data packets for off-chip processing. The first approach minimizes transmitted data and is most responsive; but embedding an additional high-performance processor and relevant subcircuits can be prohibitively expensive (for both price and power requirements), and reduces the potential for using radar data for time-series trend prediction or for multi-sensor fusion approaches, in which the outputs of multiple sensors (radar or non-radar) are combined. The second approach has the aforementioned drawback of large data storage and transmission costs, which can rapidly escalate as more radar-based sensors are deployed. A hybrid approach is therefore deemed most suitable. In this approach, a data minimization approach is initially applied, such that the radar data is first processed to find targets, then target features are analyzed either algorithmically or using lightweight machine learning classifiers to determine whether they are of interest, and subsequently transmitted off-sensor for additional processing or classification. Off-chip could mean a local gateway device, which combines and processes data from one or more sensors before transmitting the data off-site, or a local server, which is not connected to an external network, or to the Cloud.

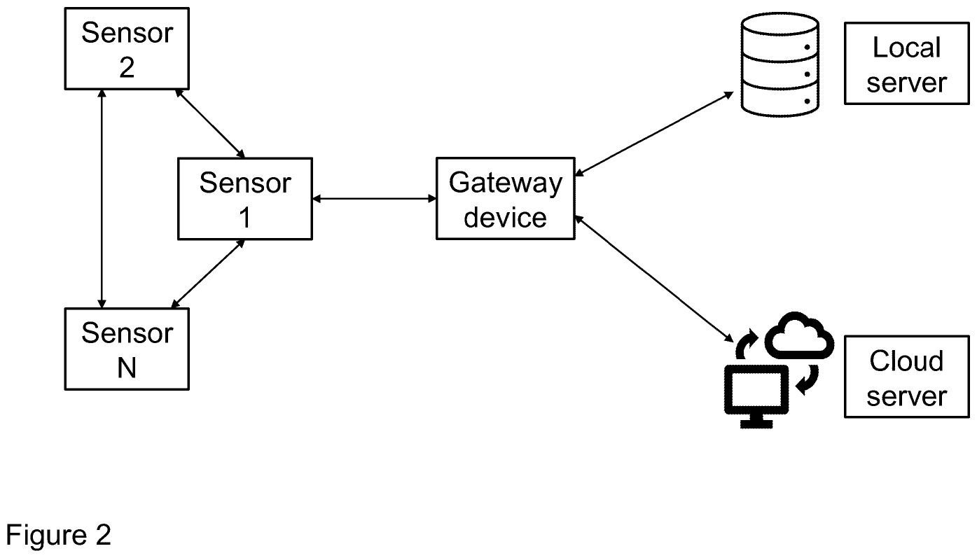

With reference to FIG. 2, a simplified block diagram with an example of the overall system architecture is shown. In this example, N sensors form a wirelessly interconnected mesh network, in which Sensor 1 serves as the root node. Data/sensor commands are propagated through the mesh network to/from Sensor 1, and on to a gateway device. Data/commands are also transmitted between the gateway device and local- or cloud-based servers. Optionally, further processing of the aggregated data may be performed on the gateway device or on the servers. The root node (Sensor 1) may also serve as the gateway device.

It is necessary to track and classify multiple targets, as otherwise the responses of two or more targets in the detection range of a sensor will lead to interference, and will lead to false readings and classification. This is particularly problematic with highly dynamic motions such as falls. Standard radar techniques are first used to find points of interest (using constant false alarm rates {CFAR} for example), which are aggregated to generate a point cloud of the scene, and to track moving targets (using the extended or unscented Kalman filters for example). Micro-Doppler signatures are characteristic responses which can be used to distinguish targets based on their micro-motions— particularly small periodic motions such as the swinging of arms and legs, the rotation of a drone's rotary blades, or the movement of a chest due to breathing and a beating heart. They are also recognized as a useful tool for identification of targets—including people, vehicles, drones and birds—and for classification of activities (including fall detection and gestures). Beamforming is first applied to the radar cube, at the range bin corresponding to the location of the primary target. The beamforming weights are determined by the angle-of-arrival of the target from the sensor, as obtained using the clustering and tracking algorithms, and have the purpose of minimizing interference due to secondary targets in other range bins and/or at other angles. The output will be the slow-time response of the target, which can then be transformed to the target's complex Doppler (velocity) spectral response via an FFT. The target's complex micro-Doppler signature is then obtained by collecting the spectral response over a series of sequential data cubes (which corresponds to a new frame of measurement time with a new timestamp). Optionally, the position that is monitored can track the position of the target, or it can remain in a fixed location. Furthermore, a window (such as a Hamming window) may be applied to the spectral profile, and the profile may also be transformed to an alternative domain, such as the wavelet domain. These variations are all encompassed by the term ‘micro-Doppler signatures’. It is further notable that the beamforming approach can be applied to each detected target so, for every time segment in which they are detected, they each have an associated micro-Doppler signature.

This micro-Doppler signature of each target, as well as other measurements, can serve as inputs to an ML algorithm. An example of this could be a convolutional neural network (CNN) or multi-layered perceptron (MLP). It is hugely beneficial to be able to analyze this information in real-time on the radar microprocessor, i.e. on the ‘edge’, and so the CNN for example is simplified to reduce the numbers of hidden layers, and to minimize the number of neurons present. Because the signatures of different actions (for example standing, sitting, walking, running, falling) are typically easily distinguishable, the ML subsystem complexity can be reduced significantly, which allows real-time ‘edge’ processing for these scenarios. Alternatively, a dictionary of classifiers based on radar parameters can be calculated for a target, such as but not restricted to positional center-of-mass, velocity, change of position or velocity, envelope of position or velocity, with these parameters serving as inputs to a lightweight ML classifier, such as but not restricted to KNN or k-means clustering. The ML subsystem may also be used to differentiate or distinguish between a person, a pet, a bed, or another object.

Additionally, the system may use any stored or collected data from one individual or from a population to predict user behavior. Examples are provided below.

Collected data may also include other known subject-related characteristics such as gender, age, health information or fitness level as well as other environmental measurements such as temperature data.

The system may also include an additional subsystem for wireless transmission (for example via Wi-Fi or Bluetooth) of encrypted radar data to a central server or cloud server. This data could be sent using one of a number of lightweight protocols, such as but not restricted to MQTT. The central server can then further process the data from a plurality of radars, send processed data to additional devices, trigger alarms, or store data for further analysis.

The wireless transmission subsystem can also be used to remotely update the configuration of the radar. Commands can be transmitted to the radar to update its configuration, whereby the configuration may include but is not restricted to the waveform configuration (for example the number of chirps per frame, the number of ADC samples per chirp, the sampling rate, the time period of each chirp), or software parameters to assist with processing (for example the orientation of the sensor, the areas or volumes of space in which to track targets, the tracking filter parameters, classification parameters, etc.). Examples of usage include: updating the radar when it is moved to a different room; updating the radar configuration to monitor areas within a room, such as desks and doorways; increasing/decreasing sensitivity of the tracking algorithm if it is missing targets/generating false detections; increasing sensitivity and updating tracking parameters for specific parts of the scene, such as through a wall to monitor an adjoining room; updating the waveform configuration or modulation scheme to improve resolution over a smaller monitoring area; updating the waveform configuration to increase sensitivity for vital signs measurements.

Response of the radar to clutter in the environment is important to its correct operation and functionality. Remote configuration of the radar enables fine-tuning of its performance in response to measurements. This can be performed manually, by updating the waveform and software parameters, or it can be performed algorithmically or through machine learning approaches. In either case, the system responds to inputs, which convey how accurately the radar is performing and subsequently updates the waveform and software parameters. These could be user inputs which may include the number of occupants detected compared to a ground truth, or the location of a false reading or missed detection, and the ground truths could be entered manually by an operator or they could be obtained through some other measurement method. In addition to the waveform parameters, the software parameters which may be updated include, but are not restricted to: number of points in a cluster to assign a track, required signal-to-noise ratio of points to assign a track, point velocity or spatial spread criteria, number of frames before a detected cluster is assigned as a track, etc. While these can be updated through defined guidelines, and specific knowledge of the system combined with user experience, a machine learning approach is suitable as it can be trained to optimize across the full set of parameters.

Multiple radars can be used to improve performance of the system. They can be used independently with their detections projected onto a single output based on the radars' positions within the global coordinate system. Alternatively, the sensor detections can be combined together, e.g. if detected point clouds are transmitted to a central server, gateway, or other processing device, then using the radars' respective positions in the global coordinate system, the point clouds can be combined, accounting for relative accuracy levels, to generate more precise clusters. Due to the fact that the radar measurements are more accurate at the antenna array's boresight, this can be used to weight the detections to more accurate readings. Optionally, a positional calibration process can be run, in which each radar sequentially transmits a known sequence (such as continuous wave, single frequency emission), while the other radars detect and localize that position. The radars will then have their relative positions within the error bounds of their measurements.

The sensors can also be connected using mesh networking technology (such as mesh Wi-Fi). This is beneficial because it: enables more efficient sharing of radar data and computational burden between the sensors, gateway devices, and other connected sensors and processors; allows for more reliable and robust connectivity; reduces burden on existing Wi-Fi networks or other communications infrastructure, which is particularly important in environments such as hospitals where connectivity may be poor, or where they are considered critical infrastructure; allows sensor coverage in areas or rooms where there is no existing networking capability, and also extends coverage to other Internet-of-Things (IoT) sensors; provides a simpler but more secure connection to external servers or clouds, as data is sent through a single node, fully encrypted and compressed.

The system may also include a sensor or sensors to detect its orientation or movements, through inertial sensors such as an accelerometer/gyroscope. This can provide notification if the sensor has been knocked over, rotated, or moved to another room. The configuration can then be updated to account for its new environment, or a notification provided for maintenance crews to reposition the sensor.

Hence the mm-wave radar system presented provides coherent wireless sensing that presents an attractive form of environmental monitoring in locations such as hospitals, office buildings, and in the home. This is because it can be used to concurrently track location and monitor activities, and there is significant opportunity to do this without the use of tags, wearables or cameras—which are inappropriate in sensitive areas such as operating theatres or restrooms.

Additionally, homecare monitoring is also one attractive proposition, particularly in the case of vulnerable residents living alone where it is important to be able to monitor for abnormal conditions, emergencies, and degradation of wellbeing, including detection falls, when the resident is in distress, and for deterioration in mobility. The mm-wave radar system provides many advantages as it can be used to monitor throughout the home, through certain walls, including in sensitive areas such as bathrooms where cameras are not suitable. The system can even operate in many occluded environments, such as through smoke, providing valuable use cases for fire safety. It also avoids the need for pendants and other wearable devices, which are only activated in a fraction of expected cases. Another related application is as support for hospital ‘virtual wards’. Virtual wards aim to keep patients out of hospitals, or accelerate the hospital checkout process, because increased lengths-of-stay are highly correlated with increased risk of infection and decompensation (a phenomenon which leads to increased recovery times). This is done by providing an environment in their home from which their health and wellbeing can be monitored remotely. This is achieved through monitoring of their movement and mobility, as well as detection of falls, and in conjunction with other sensor measurements (oxygen levels, blood pressure, temperature, etc.).

The increasing number of millimeter-wave (mm-wave) band applications—including 5G, IEEE 802.11ad/ay, the 60 GHz ISM band, and automotive radar—are of significant interest as they enable high bandwidth (and thus high resolution) sensing, small package sizes (due to the small wavelengths), and relatively low-cost devices (due to the proliferation of commercialization activities).

Examples of use cases and applications are now described.

### Fall Detection and Prevention:

Monitor personal movements over a period of time, to predict whether there is risk of falling. For example, an elderly or immobile person stands up and walks a distance a number of times over the course of a day; this data can be fitted to a model such as Timed Up and Go (TUG), or similar, to predict a trend of decreasing mobility over time. A machine learning (ML) algorithm is applied to this trend to establish features of when a person may be at risk of fall. A local care or health centre, or a previously identified care person or relative, may then be notified.
Falls are detected through use of a machine learning algorithm, such as a convolutional neural network (CNN) or multi-layered perceptron (MLP). This is applied to a micro-Doppler measurement, which plots the Doppler response of a target versus time, or a wavelet transform, of which relevant features may be detected by the ML algorithm to classify an action as a fall. This may then trigger an alert.
User mobility before, at the time of, and after the fall can be recorded for further analysis, to detect trends which may predict future falls, and also to evaluate the recovery process.
The system does not require wearables. Instead, real-time tracking logs a time-series of movements which provides a continuous stream of data for fall risk/prevention/detection analysis. This is analysed through a recurrent neural network (RNN) for example.

Vital Signs Monitoring:
The radar can track multiple users at any time, and concurrently measure vital signs (including but not restricted to heart rates and respiration rates, and their respective waveforms). These vital signs present as time series which can be analyzed, for example through an ML algorithm, to: predict physical deterioration; or identify illnesses early; or identify long-term trends. This may be done in conjunction with activity monitoring. As an example, an increase in heart rate at morning wake-up over a period of months may identify a potential illness or health deterioration. In this latter case, wake-up can be detected through tracking a person's movement on a bed and recognising the action of standing up or other actions related to waking.
The system allows tracking and measurement of vital signs of multiple targets, and also detects long-term trends through combination of vital signs detection with activity monitoring. This enables a large suite of potential investigations of health predictors, through easy, wearable-free monitoring of people during their day-to-day activities.

### Gait Analysis:

Response of scattered radar signals in association with micro-Doppler can be matched to a model of gait (typically developed around the biomechanics of a skeletal structure). This can be done using a single radar, or a plurality of radars in a room. An ML algorithm is typically used to match features from the measurements, and subsequently determine limb length, posture, movement rates of limbs, and other characteristics of the body.
Gait analysis may be used to identify changes in posture over a period of time, or to distinguish between two different people (for example a parent and their child), or to identify from within a defined subset of people (for example, the system learns to recognise the inhabitants of a home, and associate activities to them).
This implementation allows gait analysis through use of either one, or a plurality, of standalone radars with on-board, edge processing. Additionally, the analysis is used to detect long term posture changes over time, and to identify between different people.

### Gesture Recognition:

Real-time monitoring of tracked people can also be used to monitor and respond to gestures. Target micro-Doppler signatures and/or parameters are generated by the radar, and can be associated to predefined gestures through analysis by use of an ML algorithm. This may be used to raise an alarm by waving, for example.
Prior systems have applied gesture recognition at short ranges (less than a meter). In comparison, the system enables tracking of people over an entire room, and gesture recognition can be applied to multiple users at larger distances (up to five meters or more), and can be used for additional use cases, such as raising an alarm.

### Identification:

Tracking and subsequent measurement of target characteristics can be used to identify measured targets in a room. This may be done using one or more of the following: micro-Doppler signature; height (through radar measurements parallel to a person's height); speed of movement; vital signs; gait analysis. Target related characteristics are then used as inputs to an ML algorithm, which subsequently searches for specific identifying features within them, which may include but is not restricted to, limb length, or specific features of a measured or generated waveform.
Over time, the radar will learn to recognize individuals whom have been in its presence. It may also then flag unidentified persons, which may include guests to a home, or intruders. The radar may then notify the home residents, or a security company, that an unidentified person is present, and by tracking their path it may also provide information on their current location and means of entry (such as through a window). The system is able to flag unidentified occupants.
Additionally, identification and gait analysis can be used to distinguish between people and animals, which would have very different scattering profiles. For example, a dog would typically have a shorter profile perpendicular to the floor, but a wider profile parallel to the floor, and further micro-Doppler in conjunction with gait analysis would show four legs. Further, small animals such as mice can be detected and tracked, which would provide information on their route to entering a home.
Further, the invention enables target tracking using a mm-wave system, identification of the tracked target, and subsequent association of a tracked path with an identified target.

### Physical Activity Tracking:

Physical activity can be tracked, both in the short-term in the form of exercises (which may be prescribed by a doctor, clinician, or health instructor for example), or in the long-term in the form of daily activities.
Short-term tracking may be used to characterise movements, count jumps or other exercises, and determine mobility over short periods of time.
Long-term tracking can be used to compile time series which can be useful for the monitoring or detection of health trends, such as identifying long-term deterioration in physical mobility, or long-term improvements which may be outcomes of physical rehabilitation.
Long-term time series applied to large populations can also be used to search for features correlated to future falls or other physical deterioration. Additionally, for large enough populations undergoing physical rehabilitation, time series data can be used to analyse the efficacy of different workout routines or plans.

### Vision in Obscured Environments:

Smoke and haze are transparent to RF and mm-waves. The radar would therefore be operational in an obscured environment, for example in case of a fire.
This could be useful for tracking presence and counting numbers of people in rooms for notifying fire services, or tracking wellbeing of all persons in a room with poor visibility, which may be caused by solid carbon dioxide (dry ice).

### Device/Equipment Engagement

Real-time monitoring of position can be used to accurately track proximity to devices and equipment, where the device can include one of a range of commercially available voice-controlled virtual assistants. The radar can track a person's and a device's position to high precision, and output a log of when a person is within a predefined distance of the device. This can be used to present information on engagement of persons with the device, who specifically makes use of the device, timing of the device (when it is used, and for how long).
Comparison of the radar logs with a voice-activated device's logs can also identify where a user would typically be when they activate and use the device.
Control of a device through gestures, for example raising the volume of an audio system from across the room, by use of a predefined motion that would be recognised by the radar, without having to vocally speak an instruction or use a remote control.
The system provides concurrent tracking of a person and a device/object/equipment, using radar, in order to monitor user engagement with the device. Further the simultaneous tracking of multiple users across a room (up to around m), and monitoring of each user for gestures to control the device is provided.

### Acoustic Vibration Detection

Acoustic (mechanical) vibrations would modulate the radar wave, and so further processing can be used to detect acoustic frequencies in the radar's baseband. This is done by applying a bandpass filter over a small range of frequencies, and then searching for a response in within that range.
This can be used to, for example, detect the vibrations of a speaker or musical instrument to determine the output frequency, to for example verify that its response to a certain note (such as Middle C) is correct and in tune.
A filter bank can be used to reconstruct a range of acoustic frequencies, which may be used to detect speech or other audio data, as presented through vibrations through a structure, which may be a wall, a musical instrument, a speaker.
   Multi-Target Tracking and Activity Classification with Millimeter-Wave Radar

We now describe a specific example with a multi target tracking and activity classification system based on a digital beamforming approach using MIMO radar. The machine learning model is based on a Deep Neural Network (DNN) that has been configured to recognize six exercise-based classes. The system is able to achieve prediction with over 95% classification accuracy for all classes.

The system is extendable to classify any other use cases, as described in the above section, and can be applied to detection of other activities, such as fall detection.

This system presents a methodology for high accuracy tracking of multiple targets using a 60 GHz radar system, and a deep neural network (DNN) applied to the micro-Doppler response for classification of exercise activities, which are selected as demonstrators due to their mix of high- and low dynamic movements, that take place in all three spatial dimensions.

The system described provides a range resolution of about 6.4 cm and Doppler resolution of about 0.18 m/s. The system further successfully reduces interference between closely neighboring targets.

Typical system variants may provide different range resolution depending on a number of factors, such as operational bandwidth.

Measurements of individual target micro-Doppler signatures are demonstrated, even in the presence of multiple other moving targets. The signatures are used to train a Deep Neural Network (DNN) for activity classification.

A NodeNs ZERO 60 GHz IQ radar is used for all experiments presented here. For the presented experiments a bandwidth of BW=1.8 GHz is used. It operates using the principles of FMCW in conjunction with a time-division multiplexing (TDM) scheme, in which a linear chirp ramps the frequency up over N_{adc} ADC samples. These correspond to the fast time samples in the context of radar signal processing, on which a Fast Fourier Transform is performed to obtain the range profile. A value of N_{adc}=96 is used, as a tradeoff between maximum range and data volume. The radar therefore has a range resolution of

$$Δ r = \frac{c}{2 B W} = 6.3 cm ,$$

and a maximum detection range of rₘₐₓ=N_{adc}Δr=8.1 m. In slow time, the radar emits N_{chirp}=96 identical, linear chirps. Following a second FFT, this presents a maximum detectable velocity of

$$v = \frac{\pm c}{4 {f}_{0} {T}_{chirp}} = \pm 8.6 m / s$$

with a velocity resolution of

$$Δ v = \frac{2 \vert v \vert}{{N}_{chirp}} = 0.18 m / s .$$

The radar transceiver consists of an antenna array with 3 Tx transmitters and 4 Rx receivers, each with dipole-like radiation patterns, resulting in a 12-element virtual MIMO array. In this application it enables detection in two angular dimensions: azimuth (measured on the projection to the x-y plane, from the x-axis) and elevation (from the z-axis).

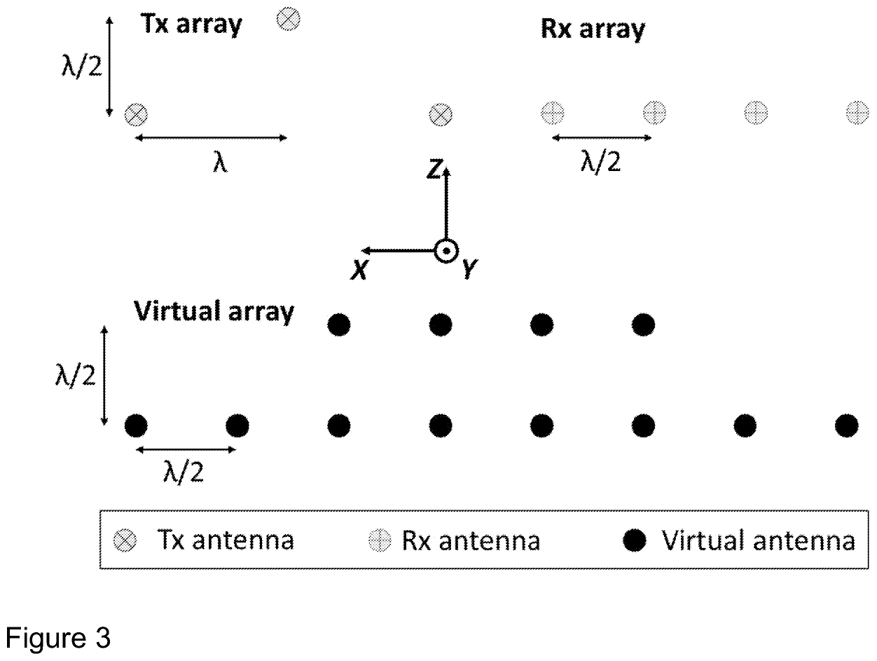

With reference to FIG. 3, a diagram of a MIMO processing subsystem is shown. The MIMO processing subsystem is used to form a 2D virtual array with 12 elements. There are two physical arrays: a 3 element transmit array and a 4 element receive array.

Digital beamforming is used to obtain the angular spectral response, \hat{P}(ϕ, θ) with high precision, although we note that the ability to distinguish between objects which are close to one another is limited by the array aperture size. Popular approaches include the Minimum Variance Distortionless Response (MVDR/Capon) beamformer, in which the total collected power is minimized in order to create nulls away from the pattern of the main search beam. The angular response is given as,

$${\hat{P}}_{M V D R} ( ϕ , θ ) = \frac{1}{{ν}^{H} {R}^{- 1} v} , \qquad ( 2 )$$

where ν is the steering vector for a specific search angle pair (ϕ, θ), R=E{xx^{H}} is the spatial covariance matrix, E{⋅} is the expected value, and x(t) is the vector of measurements at each of the 12 virtual antennas. Other suitable approaches exist, such as MUltiple SIgnal Classification (MUSIC) which detect sources by searching the noise subspace, however this comes at the cost of additional computation complexity and some knowledge of the number of targets. For an antenna at position (x,z)=(nλ/2, mλ/2), the steering vector element is

νₙₘ(ϕ,θ)=e^{-jπ[(n-1)sin ϕ][(m-1)sin θ]}. (3)

The radar scans the environment at 20 Hz—which is sufficient to capture most human micro-motions—and associates a timestamp to each scan. The receivers are coherent and measure the complex IQ parameters. The phase sensitivity enables detection of minute motions. This is because one wavelength, λ≈5 mm, perpendicular to the plane of the array corresponds to a phase rotation of 2π, and so even small movements can be detected through phase shift measurements. A phase shift of Δφ=36°, for example, will correspond to a movement of 0.5 mm.

Once the radar cube—which consists of the complex-valued IQ data corresponding to each range, azimuth, elevation and Doppler bin—is calculated, the aim is then to identify relevant subjects in the scene, locate their positions, and then classify their actions. The radar cube, however, is large and not suitable for real-time processing, and so a detection algorithm is necessary to identify areas of interest. The output of the detection algorithm is a point cloud, which is a list of points corresponding to range, azimuth, elevation and velocity. The naïve method is to simply poll the amplitudes of the response at each spatial bin (r,ϕ,θ), assigning a point to each bin if it exceeds a certain threshold value. Alternatively, the Constant False Alarm Rate (CFAR) algorithm is more robust to noise. On selecting a list of points, with their associated range and angles, the velocity is subsequently defined as:

ν=argmax|ν|, (4)

where ν is the vector representing the Doppler spectrum at that spatial point.

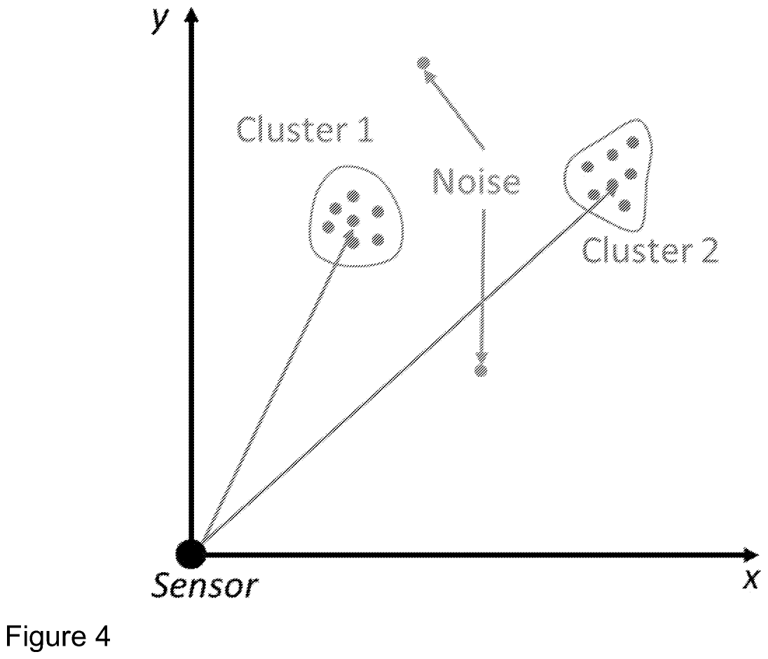

With reference to FIG. 4, a DBSCAN algorithm is illustrated including two clusters. The DBSCAN algorithm is used to cluster points together, or to label them as noise if they are not associated with a cluster. Two clusters are detected and two points are labelled as noise.

Targets are then identified through application of the DBSCAN clustering algorithm to the point clouds, such that each point will be assigned either to a specific cluster or as noise, as shown. Each cluster m is then associated to a subject, and will have a corresponding center-of-mass (xₘ,yₘ,zₘ), which is the position of the target with respect to the radar. The nearest appropriate range bin and azimuthal bin is used for 2D tracking, and an additional elevation bin for 3D tracking. An experiment was performed with two subjects near one another, and both at a range of 2 m from the radar. The subjects alternately performed dynamic actions (jumping up-and-down in this case). It is important to be able to distinguish between the actions of different users, so as not to confuse an activity classification system with dynamic noise. A potential use case is an environment with thin wall, which may be transparent to RF and mm-waves, in which dynamic motions of a resident in an adjoining room may mask detection of a fall in the primary room, because the Doppler responses are convolved if appropriate processing is not performed. The presented approach will avoid this scenario.

A significant challenge in using Doppler responses for activity classification is the cross-contamination of a target's signature with that of a nearby target. This is particularly challenging for targets at similar distances from the radar.

To mitigate this, spatial filtering is used to isolate each target from nearby targets. Analogously to the angle-of-arrival estimations, the corresponding beamforming weights associated with each antenna are calculated using the angular response from Eq. (2). The weighting vector is given by

w=P_{MVDR}(ϕ,θ)·R⁻¹ν(ϕ,θ). (5)

If we consider that the time interval between chirps is t_{c}, then the spatially-filtered response of the target at range r and azimuth angle ϕ, to chirp p is

zₚ=z(pt_{c})=w^{T}(ϕ)·x(r). (6)

The Doppler spectrum at time T is subsequently calculated as the discrete Fourier transform of z, i.e.

Z(T)=DFT(z). (7)

On initial detection of a target, its center-of-mass (xₘ,yₘ) is monitored continuously for a period of time (1.5 s corresponding to 30 frames). The micro-Doppler signature is a time-frequency plot which shows the evolution of the Doppler spectrum Z with time. Note that, whilst it is also possible to update the location to the target's recalculated CoM at each frame, we use a fixed location as this maintains a consistent phase reference, which aids in fine-motion detection (including for vital signs monitoring).

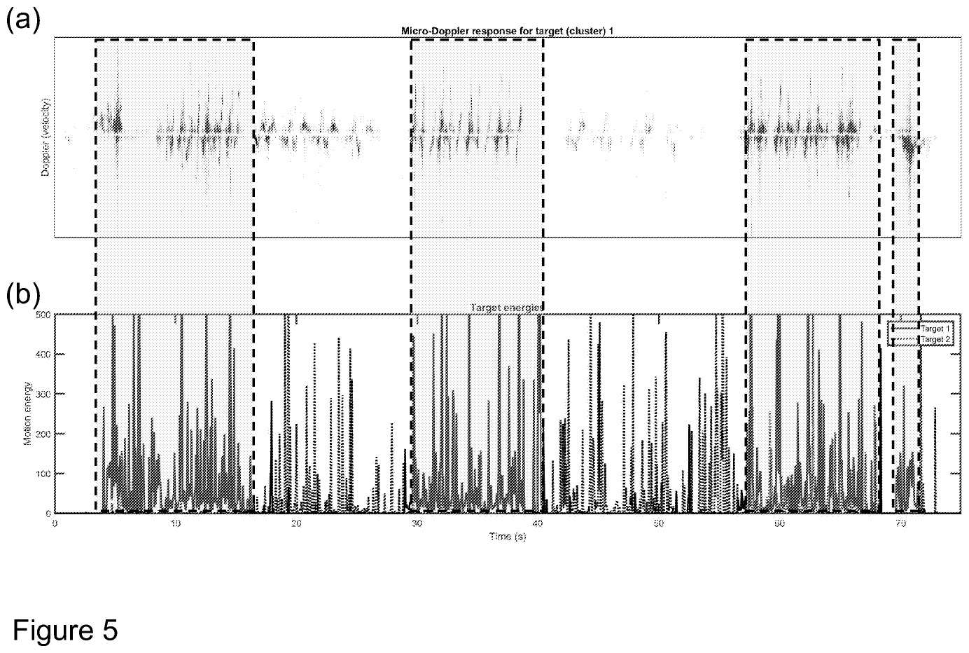

FIG. 5 illustrates the efficacy of the beamforming approach in separating responses of two equidistant targets. These targets are around 1 m apart, and are both at a range of 2 m from the radar (i.e. in the same range bin). They alternate in moving dynamically (jumping). The shaded areas correspond to Target 1 jumping while Target 2 is stationary. In order to accurately classify activities of a target, interference from other targets must be minimized. Beamforming enables separation of both targets, to minimize interference in their responses. In FIG. 5 (a) the micro-Doppler response of Target 1 is shown. The unshaded areas, in which only Target 2 is moving, shows relatively little activity. Velocities (y-axis) are in range ±5.8 m/s. In FIG. 5 (b) The motion energies of both targets, calculated as ΣₙVₙ², where νₙ is the velocity of a point n that belongs to a target. The shaded areas correspond to movement by Target 1. As the targets are equidistant from the radar, without beamforming there would be significant interference between their responses, and the micro-Doppler response of Target 1 would show significant activity in the unshaded areas. This therefore demonstrates the efficacy of the beamforming approach.

The figure shows the efficacy of the method, wherein even two very closely spaced targets have relatively little contribution to the other's micro-Doppler signature.

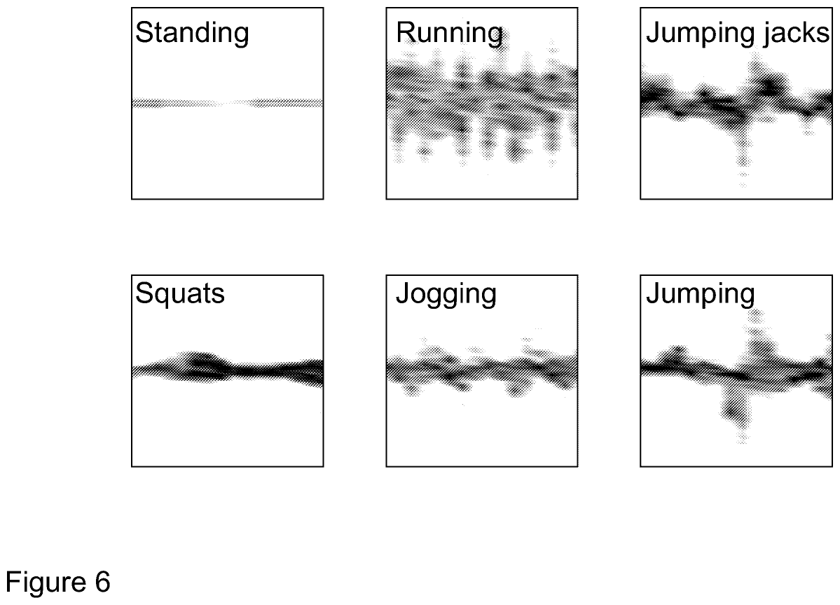

FIG. 6 shows the micro-Doppler signatures obtained over a 1.5 s interval, for six different activities each performed with the subject remaining in one location (clockwise from top left): standing, running, jumping jacks, jumping, jogging, squats. The signatures each show characteristic features specific to each activity, which can be used to classify the activity, and also to extract analytics based on motion intensity and frequency. The vertical axes correspond to radial velocity, and are scaled to ±5 m/s, while the horizontal axis is time over a range of 1.5 s.

Bilinear interpolation is used to smooth the plots, which helps to highlight features. The 0-Doppler bin (center of the vertical axis) is set to 0, which is equivalent to subtracting out the mean value of the samples. This is a commonly used radar processing technique used to suppress stationary objects, and in particular to mitigate against the effects of clutter. From the plots, the Standing signature is clearly evident with any motion restricted to the near-0 bins. Squats involve relatively slow vertical movements, and so are similarly restricted to the near-0 Doppler bins. Running (performed in one spot) is clearly the most dynamic motion.

There is a significant and growing body of work on using machine learning techniques for classification of activities based on their Doppler responses. The primary innovations here are the approaches for distinguishing between multiple moving targets and their respective activities. A deep neural network (DNN) trained using a transfer learning approach is used to classifying human activities. The SqueezeNet DNN is used as it is lightweight, requiring <0.5 MB of memory and 50× fewer parameters than comparable networks such as AlexNet, while maintaining similar accuracy. SqueezeNet was originally trained for computer vision, with around 1,000 classes. It is used here for the classification of six activities using a training set of 1,283 micro-Doppler signatures, with 315 (i.e. 80%-20% training-validation sample split) signatures kept for validation. The overall classification accuracy is over 99%, with only some miss-classifications between running and jumping jacks, which are highly-dynamic movement.

Hence a method for tracking and activity classification of multiple targets using mm-wave radar has been provided, coupled with MIMO radar and beamforming techniques. The DBSCAN clustering algorithm is used to identify targets of interest, and to isolate potential noise detections. This can be extended with a tracking algorithm (such as the Unscented Kalman Filter) to improve tracking precision of moving targets.

Beamforming is then used to digitally reduce the field-of-view of the array to focus on each of the targets, in order to minimize clutter and movements from other targets. Imaging techniques combined with multi-radar fusion can be used to further improve accuracy. The micro-Doppler signatures of the targets are then measured, and are used in conjunction with a Deep Neural Network built upon an AlexNet transfer learning model. This shows excellent performance. We note that in future a one-class classifier can be used to discard noisy movements, which may be misidentified as one of the six trained classes, and the. classification can be further improved by accounting for motion in the (X, Y, Z) planes, rather than using just the velocity response.

### APPENDIX 1— KEY FEATURES OF THE MM-WAVE RADAR SYSTEM

The present invention offers solutions for the use of a mm-wave radar system for short and long-term tracking of subject activity or behavior such as physical activity, as well as for determining or predicting long-term health and wellbeing trends. Using machine-learning techniques, the mm-wave radar system converts a stream of radar data or backscattered signals into meaningful data outputs which near instantaneously describe the activity of multiple targets within an environment. The data outputs may then be used for immediate analysis of an environment and its occupants in real time. Additionally, they may also be used to determine whether further radar data should be sent for subsequent analysis. The techniques presented are particularly useful in a homecare environment, where the knowledge in near real time of a person's location and current state is of benefit to determine their wellbeing and/or physical fitness.

Advantageously, tracking may be done tag-free (with no wearable device). Tag free use cases are particularly attractive as wearables are uncomfortable to many people, are subject to being lost, and require frequent charging. A wearable-free system provides continuous wellbeing and security monitoring without requiring a user to remember to charge or put on the device.

There are no limitation on the number of targets and their associated activity, provided that the number of sensors used may be increased depending on the environmental configuration.

In the following sections, we outline key features of the mm-wave radar system; we list also various optional sub-features for each feature. Note that any feature can be combined with one or more other features; any feature can be combined with any one or more sub-features (whether attributed to that feature or not) and every sub-feature can be combined with one or more other sub-features. The invention is however defined in the appended claims.

Feature 1: Multi-Target Classification Using Machine Learning

In one implementation, a mm-wave radar system enables the classification of multiple activities from multiple subjects or targets using a machine learning model. The received radar data or backscattered signal is filtered using digital beamforming in order to determine a micro-Doppler response of at least one target. The micro-doppler responses of one or more targets are then used as input to a machine learning engine for classification purposes. Hence, the system is able to convert a stream of backscattered signals into data outputs or activity records that describe multiple targets. The data outputs are configured to be meaningful such that instantaneous information related to the activity or state of multiple targets in an environment can be used to provide real-time information related to multiple targets. Further, the data outputs can also be used to determine whether specific radar data should be transmitted for subsequent data analytics. Target classification may include for example: identification, whether they are human/non-human, whether they are an adult or child, whether they are in a wheelchair. Activity classification may include for example: walking, running, jumping, exercising, sitting, lying down, standing, falling over, sleeping or any other activities.

We can Generalize as Follows:

A mm-wave radar system for determining an activity record from multiple targets comprising:
i) at least one sensor configured to transmit a mm-wave signal waveform and to receive backscattered signals from multiple targets; and
ii) one or more processors configured to a) process the received backscattered signals, b) determine radar data related to each target, and c) process the radar data of each target using a machine learning engine that outputs an activity record related to each target, in which each activity record is linked to a timestamp.

Backscattered Signals
backscattered signals are represented as a 3d radar data cube.
stream of backscattered signals is received by the sensor and represented by a series of 3D radar data cubes, each linked to a specific timestamp.
3D radar data cube is processed using a spatial filtering based approach in order to determine the radar data associated with each target.
spatial filtering-based approach combines a range bin approach and a digital beamforming approach.
a single range bin associated with a specific target is monitored to spatially filter in the range domain.
a fast-time response of the 3D radar data cube is processed to derive a range profile, which consists of a series of range bins, from which a specific range bin corresponding to the distance between the target and the sensor is then selected to spatially filter in a range domain, which may be referred to as a range bin approach.
The spatial filtering based approach is used to isolate each target from nearby targets.

Radar Data
radar data related to a target includes one or more of the following: 3D radar data cube, micro-Doppler parameters or signature, point cloud of scene, points assigned to the target by a clustering algorithm.
micro-Doppler parameters are derived from a cluster of points assigned to the target by a clustering algorithm and from the properties of each of the points, including location, velocity, signal-to-noise ratio, as well as mathematical operations performed on those properties over a series of timestamps.

Activity Record
activity record defines metadata or parameters for the detected target.
activity record is a record of events or activities related to the detected target and associated with a series of timestamps.
timestamp includes a start value that indicates the beginning of the event or activity.
timestamp includes a stop value that indicates the end of the event or activity.
timestamp includes a series of start, stop, range values.
an event or activity is associated with a vital sign or other physiological parameters.
activity record is sent to a remote server for subsequent analysis.

Output
mm-wave radar system is configured to send the activity record to a dashboard or application or a web page.
mm-wave radar system is configured to output a digital representation of each target within an environment.
mm-wave radar system is configured to display the digital representation of each target on a dashboard or application or a web page.

ML Engine
ML engine takes the radar data as input.
a training dataset used to train the machine learning engine uses existing pre-labelled radar data.
ML engine is trained to classify an event or activity.
ML engine is trained to estimate a vital sign or other physiological parameter.
ML engine is also configured to differentiate or distinguish multiple targets.
ML engine is configured to extract features related to the target such as limb length, posture, movement rates of limbs or any other related parameters.
ML engine is configured to predict physical change (such as deterioration), or to identify abnormality or pathology (such as illnesses early).
ML engine is configured to identify long-term trends.
hybrid approach is used in which a human expert is able to manually review and classify radar data in a way that it is used to train the machine learning engine.
ML engine is located on the sensor, or in a hub or gateway connected to the sensor, or distributed across any permutation of these.

Vital Signs
The mm-wave radar system is also configured to determine a vital sign or other physiological parameter related to the target, based on the analysis of the radar data.
The mm-wave radar system is also configured to monitor the vital signs of one or more people in bed, and also monitors motions, which can be used to diagnose sleep quality.

Other Applicable Optional Features
mm-wave radar system includes a communication subsystem that is configured to send or receive encrypted radar data or command or telemetry data to a remote server.
mm-wave radar system includes multiple sensors that each scan individually or that scan different sections of an environment, in order to show the target moving through the environment.
the mm-wave radar system includes multiple sensors, in which the aggregated data of the multiple sensors is processed together.
the multiple sensors are wirelessly connected.
environment includes one or more indoor area with walls.
when a specific event or activity is detected, the mm-wave radar subsystem is configured to send an instruction or alert to an application running on a connected device.
mm-wave radar system is configured to predict a next event or activity of the detected target or predict a subsequent location of the detected target.
mm-wave radar system is configured to send an instruction or alert depending on an abnormal event or change of behavior.
mm-wave radar system is configured to send an instruction or alert depending on a predicted event or activity.
mm-wave radar system is configured to send an instruction or alert together with information or metadata that captures a specific event, such as abnormal event or change of behaviour.
mm-wave radar system does not output the entire stream of received backscattered signals.
mm-wave radar system outputs a selected portion of the received backscattered signals based on a detected event or activity.
mm-wave radar system automatically stops tracking and/or monitoring an environment when zero occupancy is detected.
the sensor includes an inertial sensor such as an accelerometer or gyroscope, such that a change of position of the sensor is automatically detected.
target includes people, animal or moving objects like wheelchairs.

Feature 2: Combination of Edge- and Cloud-Classification
The system implements machine learning techniques, either fully or partially at the edge. Edge classification includes initial classification of the data (unfiltered received data from one or multiple receivers) to identify interesting features, which serves to minimize the amount of data that is transmitted. A local gateway device can also include more powerful AI processors, including CPUs, GPUs, TPUs, etc. This can be used for immediate and responsive classification using neural networks and other computationally-intensive machine learning approaches. Data is then sent to the cloud to perform further classification and prediction.

We can generalize as follows:

A mm-wave radar system for detecting an activity record from multiple targets comprising:
i) at least one sensor configured to transmit a mm-wave signal waveform and to receive backscattered signals from multiple targets; and
ii) one or more processors configured to a) process the received backscattered signals, b) determine radar data related to each target, and c) process the radar data of each target using a machine learning engine that outputs an activity record related to each target, in which each activity record is linked to a timestamp,
in which the ML engine is located on the sensor, or in a hub or gateway connected to the sensor, or distributed across any permutation of these.

Optional Features
depending on the activity record, the mm-wave radar system is configured to output a selected portion of radar data to a remote server for subsequent analysis.
radar data is encrypted before sending it to the remote server.
selected portion is associated with a range of timestamp.

Feature 3: Remote Configuration

The configuration of the mm-wave radar system can be automatically or manually updated based on the analysis of the radar data. The configuration of the radar includes control of the radar waveform parameters (including chirp, ADC sampling rate, waveform duration, sampling time, etc.), and software parameters (tracking filter {Kalman} parameters, monitoring zones, and further processing parameters such as CFAR, etc.). Configuration updates are sent wirelessly to the sensor, e.g. through Wi-Fi, and are communicated through an application that is running on a gateway hub, a server, or on the Cloud. Configuration changes can be entered manually by a user, such as: to define the monitoring/room area when a sensor is first installed, or to specify specific location such as desks which should be monitored; or it can be done programmatically. In the latter case, the user provides inputs which can include: too many false target detections, not enough detections, detections from neighbouring rooms, improved sensitivity required in a certain area (such as through a wall). These inputs can be processed through a machine learning (e.g. reinforcement learning) to update the configuration to achieve the desired performance.

It is hugely beneficial to be able to adaptively recalibrate and reconfigure a radar to optimize performance, especially when trying to monitor moving targets as dynamic scenes with a lot of motion leads to clutter and noise or when complex indoor environments are monitored.

We can generalize as follows:
a mm-wave radar system for detecting an activity record from multiple targets comprising:
   i) at least one sensor configured to transmit a mm-wave signal waveform and to receive backscattered signals from multiple targets; and
   ii) one or more processors configured to a) process the received backscattered signals, b) determine radar data related to each target, and c) process the radar data of each target using a machine learning engine that outputs an activity record related to each target, in which each activity record is linked to a specific timestamp;
   and in which the mm-wave radar system includes configuration parameters that can be remotely updated.

Optional Features
mm-wave radar system is configured to automatically detect when the configuration parameters of the mm-wave radar system need to be updated.
configuration parameters include one or more of the following: the radar waveform parameters (including chirp, ADC sampling rate, waveform duration, sampling time, etc.), and software parameters (point clustering parameters, tracking filter {Kalman} parameters, monitoring zones, and further software-defined processing parameters such as for CFAR, etc.), location of the sensor within an environment or specific area of interest.
configuration parameters further include information related to the room or environment that needs to be monitored, such as floor plans, specific locations of objects, specific areas of interest.
configuration parameters further include specific activity record or event or activity to be detected, and number of targets of interest.
configuration parameters further include parameters related to a virtual area.
virtual area is generated by a software module to define a specific area of interest.
mm-wave radar system includes an application [[or webpage]] running on a gateway hub, server, or cloud that is configured to wirelessly send the configuration parameters to the sensor.
configuration parameters are stored on the gateway hub, server or cloud on which the application is running.
configuration parameters are entered manually by a user.
mm-wave radar system is configured to apply different configuration parameters in different areas.
mm-wave radar system is configured to automatically determine optimum configuration parameters for an environment or specific area of interest.
human expert is able to manually review data outputted by the machine learning engine in a way that it is used to train the machine learning engine to improve the configuration parameters.
mm-wave radar system is configured automatically indicate when the sensors are correctly or incorrectly positioned.
mm-wave radar system is configured automatically indicate a dead area, in which a dead area refers to an area that the mm-wave radar sensor cannot correctly scan.
mm-wave radar system is configured automatically indicate when the sensors correctly or incorrectly positioned to detect specific event or activity or vital signs or other physiological data.

Feature 4: Detection of Abnormal or Change of Behavior of a Subject, Such as Mobility Deterioration

The mm-wave radar system logs activities of an occupant (e.g. in the home) over a period of time (days, weeks, months). The mm-wave radar system uses activity classification to identify when a person performs activities including: standing up, walking across a room, sitting down, etc. The radar detects the location of the occupant, and in combination with timestamps, a record is generated of how long it takes for the occupant to perform defined activities, such as: getting up from a sofa and walking to the kitchen, or walking from a bedroom to a bathroom. The trend of this data is measured over a period of time to generate a time-series, at which point it can be referred to by a physician to diagnose whether a person's mobility is improving or degrading. A recurrent neural network (RNN) or other machine learning techniques can be applied to this data to identify mobility trends, to predict how mobility will change, or to predict if a person is at risk of falling (degradation in mobility is a leading indicator of potential fall risk).

We can generalize as follows:
a mm-wave radar system for detecting an activity record from multiple targets comprising:
   i) at least one sensor configured to transmit a mm-wave signal waveform and to receive backscattered signals from multiple targets; and
   ii) one or more processors configured to a) process the received backscattered signals, b) determine radar data related to each target, and c) process the radar data of each target using a machine learning engine that outputs an activity record related to each target, in which each activity record is linked to a timestamp;
   and in which the mm-wave radar system is configured to detect an abnormal event or change of behavior.

Optional Features
mm-wave radar system is configured to send an instruction or alert together with the activity record that captures a specific detected event, such as abnormal event or change of behaviour.
mm-wave radar system is configured to integrate with audio monitoring sensor that automatically turns on when a specific event or activity is detected, such as a fall.
audio monitoring sensor automatically plays voice message when a specific event or activity is detected, such as a fall (i.e voice message could be “help in on the way”).

Note

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred example(s) of the invention, it will be apparent to those of ordinary skill in the art that numerous modifications can be made without departing from the principles and concepts of the invention as set forth herein.

1. A mm-wave radar system for detecting an activity record from multiple targets comprising:
i) at least one sensor configured to transmit a mm-wave signal waveform and to receive backscattered signals from multiple targets; and
ii) one or more processors configured to a) process the received backscattered signals, b) determine radar data related to each target, and c) process the radar data of each target using a machine learning (ML) engine that outputs an activity record related to each target, in which each activity record is linked to a timestamp; in which the activity record is a record of events or activities related to the detected target and associated with a series of timestamps;
and in which the mm-wave radar system is configured to predict a next event or activity of the detected target or predict a subsequent location of the detected target or identify an abnormal event or change of behavior.

2-3. (canceled)

4. The mm-wave radar system of claim 1, in which radar data related to a target includes one or more of the following: 3D radar data cube, micro-Doppler parameters or signature, point cloud of scene, points assigned to the target by a clustering algorithm; and in which micro-Doppler parameters are derived from a cluster of points assigned to the target by a clustering algorithm and from the properties of each of the points, including location, velocity, signal-to-noise ratio, as well as mathematical operations performed on those properties over a series of timestamps.

5-7. (canceled)

8. The mm-wave radar system of claim 1, in which the mm-wave radar system is also configured to determine a vital sign or other physiological parameter related to the target, and in which an event or activity is associated with a vital sign or other physiological parameters.

9. The mm-wave radar system of claim 1, in which the mm-wave radar system is configured to send the activity record to a dashboard or application or a web page, and in which the mm-wave radar system is configured to output a digital representation of each target within an environment.

10-16. (canceled)

17. The mm-wave radar system of claim 1, in which the ML engine is configured to extract features related to the target such as limb length, posture, movement rates of limbs or any other related parameters.

18. The mm-wave radar system of claim 1, in which the ML engine is configured to predict physical change or to identify abnormality or pathology.

19. The mm-wave radar system of claim 1, in which the ML engine is configured to identify long-term trends.

20. (canceled)

21. The mm-wave radar system of claim 1, in which a lightweight ML classifier that is located on the sensor is first used to determine if a selected portion of radar data should be transmitted off-sensor for additional processing or classification, such as to a gateway connected to the sensor, or to a server connected to the sensor or gateway, or distributed across any permutation of these.

22. (canceled)

23. The mm-wave radar system of claim 1, in which the mm-wave radar system is configured to monitor the vital signs of one or more people in bed, and also monitors motions, which can be used to diagnose sleep quality.

24. The mm-wave radar system of claim 1, in which the mm-wave radar system includes a communication subsystem that is configured to send or receive radar data or command or telemetry data to or from a remote server.

25. The mm-wave radar system of claim 1, in which the mm-wave radar system includes multiple sensors that each scan individually or that scan different sections of an environment, in order to show the target moving through the environment.

26-27. (canceled)

28. The mm-wave radar system of claim 25, in which the environment includes one or more indoor area with walls.

29. The mm-wave radar system of claim 1, in which when a specific event or activity is detected, such as an abnormal event or change of behavior, the mm-wave radar subsystem is configured to send an instruction or alert to an application running on a connected device.

30-33. (canceled)

34. The mm-wave radar system of claim 1, in which the mm-wave radar system does not output the entire stream of received backscattered signals and only outputs a selected portion of the received backscattered signals based on a detected event or activity.

35. (canceled)

36. The mm-wave radar system of claim 1, in which the sensor includes an inertial sensor such as an accelerometer or gyroscope, such that a change of position of the sensor is automatically detected.

37-40. (canceled)

41. The mm-wave radar system of claim 1, in which the mm-wave radar system includes configuration parameters that can be remotely updated; and in which the configuration parameters include one or more of the following: the radar waveform parameters such as chirp, ADC sampling rate, waveform duration or sampling time, and software parameters such as point clustering parameters, tracking filter parameters, monitoring zones, and information related to the room or environment that needs to be monitored, such as locations of sensors within an environment or specific area of interest, floor plans, specific locations of objects, specific areas of interest and specific activity records or events or activities, or number of targets of interest that are to be detected.

42-45. (canceled)

46. The mm-wave radar system of claim 41, in which the configuration parameters further include parameters related to a virtual area, and in which the virtual area is generated by a software module to define a specific area of interest.

47. (canceled)

48. The mm-wave radar system of claim 41, in which the mm-wave radar system includes an application running on a gateway hub, server, or cloud that is configured to wirelessly send the configuration parameters to the sensor.

49. The mm-wave radar system of claim 41, in which the configuration parameters are stored on the gateway hub, server or cloud on which the application is running.

50. (canceled)

51. The mm-wave radar system of claim 41, in which the mm-wave radar system is configured to apply different configuration parameters in different areas.

52. The mm-wave radar system of claim 41, in which the mm-wave radar system is configured to automatically determine optimum configuration parameters for an environment or specific area of interest.

53. (canceled)

54. The mm-wave radar system of claim 1, in which the mm-wave radar system is configured to automatically indicate when the sensors are correctly or incorrectly positioned.

55. The mm-wave radar system of claim 1, in which the mm-wave radar system is configured to automatically indicate a dead area, in which a dead area refers to an area that the mm-wave radar sensor cannot correctly scan.

56. The mm-wave radar system of claim 1, in which the mm-wave radar system is configured to automatically indicate when the sensors are correctly or incorrectly positioned to detect specific events or activities or vital signs or other physiological data.

57. The mm-wave radar system of claim 1, in which the mm-wave radar system is configured to detect an abnormal event or change of behavior and in which the mm-wave radar system is configured to send an instruction or alert together with the activity record that captures a specific detected event, such as abnormal event or change of behaviour.

58. (canceled)

59. The mm-wave radar system of claim 1, in which the mm-wave radar system is configured to integrate with other sensor devices, such as an audio monitoring sensor, that automatically turns on when a specific event or activity is detected, such as a fall.

60. (canceled)

61. A method for detecting an activity record from multiple targets using a mm-wave radar system, the method including the steps of:
i) transmitting a mm-wave signal waveform using at least one sensor;
ii) receiving backscattered signals from multiple targets; and
iii) at one or more processors, a) processing the received backscattered signals, b) determining radar data related to each target, and c) processing the radar data of each target using a machine learning (ML) engine that outputs an activity record related to each target, in which each activity record is linked to a timestamp, in which the activity record is a record of events or activities related to the detected target and associated with a series of timestamps;
and in which the mm-wave radar system is configured to predict a next event or activity of the detected target or predict a subsequent location of the detected target or identify an abnormal event or change of behavior.

62. (canceled)

63. The mm-wave radar system of claim 1, in which the system is configured to:
i) analyse how long it takes for a person to perform a defined activity;
ii) generate a metric of long-term health or mobility trends; and
iii) determine whether a referral to a third party, such as a health practitioner is needed.
